(19) United States
(12) Patent Application Publication
(10) Pub. No.: US 2012/0164195 A1
(43) Pub. Date: Jun. 28, 2012

(54) Cosmetic Films

(75) Inventors: Tao Zheng, New City, NY (US); Leona G. Fleissman, Ridgewood, NJ (US); Xiaolan Wei, Suffern, NY (US)

(73) Assignee: AVON PRODUCTS, INC., Suffern, NY (US)

(21) Appl. No.: 13/307,722

(22) Filed: Nov. 30, 2011

Related U.S. Application Data
(60) Provisional application No. 61/426,140, filed on Dec. 22, 2010.

(51) Int. Cl. A61K 8/73; A61K 8/81; A61K 8/84; A61K 47/38; A61Q 19/02; A61Q 1/02; A61K 9/00; A61K 8/02; A61Q 1/00

(52) U.S. Cl. 424/401; 424/62; 424/642; 514/781; 514/772.5; 514/772.4; 514/779; 514/772.7; 514/777; 514/772.6; 424/63; 424/400

(57) ABSTRACT

Personal care products, including cosmetics, are provided as solid, water-soluble polymeric films which adhere to a human integument when wetted. The invention also relates to such films having plural laminated layers, at least two of which possess different cosmetic or therapeutic characteristics.

### CROSS REFERENCE TO RELATED APPLICATIONS

This application claims priority benefit, under 35 U.S.C. §119(e), of U.S. Provisional Patent Application No. 61/426,140, filed Dec. 22, 2010, the contents of which application are hereby incorporated by reference in their entirety.

### FIELD OF THE INVENTION

The present invention relates generally to solid, water-soluble polymeric films for cosmetic application to a human integument. The invention also relates to such films having plural laminated layers, at least two of which possess different cosmetic or therapeutic characteristics.

### BACKGROUND

Water dissolvable solid films have been used to improve the portability and convenience of use of certain personal care products that traditionally existed only in liquid form, the most notable example being the Listerine® Breath Strips. These types of products comprise a water-soluble polymeric matrix composed of a natural or synthetic polymer such as pullulan. To date, there have been no commercially successful efforts cosmetic products based on this technology.

PCT Patent Pub. No. WO 2009/006214A1 published Jan. 8, 2009, the disclosure of which is hereby incorporated by reference in its entirety, describes personal care dissolvable films, including for cosmetic use, comprising a water soluble film forming agent, a cosmetically acceptable plasticizer, and a thickener. However, that publication does not recognize the range of novel and surprising effects that can be achieved using cosmetic films, nor does it describe films having plural laminated layers.

It is therefore an object of the present invention to provide cosmetic films that adhere to a human integument and provide cosmetic and/or therapeutic benefits that heretofore have been lacking or difficult to achieve. It is a further object of the invention to provide cosmetic films comprising plural laminated layer of water-soluble polymer, which films adhere to a human integument to provide a cosmetic or therapeutic benefit.

### SUMMARY OF THE INVENTION

In accordance with the foregoing objectives and others, the present invention provides cosmetic compositions in the form of solid films that can be wetted and adhered to the skin to achieve a number of cosmetic benefits including, without limitation, skin tightening, delivery of active agents, and optical effects such as UV blocking and absorption, fluorescence emission, optical blurring, soft focus, color changing at different angles, and color changing at different temperatures, to name a few.

In one aspect, a personal care product is provided having the form of a solid film comprising a water-soluble polymeric matrix having an optical component homogenously dispersed therein, the optical component being selected from the group consisting of liquid crystals, glitters, optical brighteners, cosmetic fibers, and combinations thereof, wherein said solid film is capable of being wetted and adhered to a human integument.

In another aspect, a personal care product is provided having the form of a solid film comprising a water-soluble polymeric matrix, wherein the solid film is capable of being wetted and adhered to a human integument; and wherein the film includes a polymeric film former that shrinks on drying such that the film contracts in one or more dimensions when dried on the skin to thereby provide a tightening effect to the skin.

In yet another aspect, a personal care product is provided having the form of a solid, transparent film comprising a water-soluble polymeric matrix having an optically transparent dispersion of zinc oxide particles homogenously dispersed therein, wherein the solid film is capable of being wetted and adhered to a human integument to leave a transparent film thereon. The dispersion of zinc oxide particles may be of any particle size and distribution provided that the dispersion are transparent in visible light. In one implementation, the zinc oxide particles have a median particle diameter (D₅₀) between about 1 micron (μm) and about 5 μm and a D₉₀ of less than about 10 μm, on a volume basis. In another implementation, the dispersion of zinc oxide particles has a D₅₀ between about 1.5 μm and about 3 μm; a D₉₀ of less than about 6.5 μm; and a D₁₀ greater than 0.1 μm, on a volume basis. The zinc oxide particles will typically comprise from about 1% to about 30% of the weight of said film and the levels can be adjusted to provide the desired sun protection factor (SPF), for example, at least about 8, at least about 15, at least about 30, at least about 45, or at least about 60.

The personal care products may comprise any water-soluble polymeric materials that are capable of providing a solid matrix throughout which other cosmetic components, for example pigments, and therapeutic components, for example anti-aging or anti-acne actives, can be dispersed. In some implementations, the water-soluble polymeric matrix comprises a polymer selected from the group consisting of hydroxyethyl cellulose, hydroxypropyl cellulose, hydroxypropylmethyl cellulose, carboxymethyl cellulose, sodium carboxymethyl cellulose, methyl cellulose, polyvinyl pyrrolidone, polyvinyl alcohol, sodium alginate, polyethylene glycol, polyethylene oxide, xanthan gum, tragacanth gum, guar gum, acacia gum, gum arabic, pullan, carrageenan, polyacrylic acid, methyl(meth)acrylate copolymers, carboxyvinyl copolymers, and combinations thereof. Water-soluble cellulose ethers such as hydroxypropyl methylcellulose and/or polyethylene oxides are preferred.

The films may include other components to adjust the properties of the film, such as the dissolution rate, tensile strength, pliability, and the like, in particular, a plasticizer may be included in an amount from about 0.01% to about 40.0% of the weight of the film, and a thickener may be included in an amount from about 0.01% to about 10.0% of the weight of said film. The film may be any thickness depending on the desired application, but are typically between about 1 and about 5 mils in thickness.

In yet another aspect of the invention, a personal care product is provided having the form of a solid film comprising plural laminated layers, including a first layer of a first solid water-soluble polymeric matrix, and a second layer of a second solid water-soluble polymeric matrix, wherein said solid film is capable of being wetted and adhered to a human integument. The first and second layers may be the only layers of the film, in which case they are laminated together, preferably in superposition. The film may also include additional layer separating the first and second layers, and/or laminated to either face of the first and/or second layers.

The first and second layers may be the same or different, in which case they will have at least one different characteristic. For example, the first and second layers may have different refractive indices. In one embodiment, the first layer is transparent and the second layer is translucent, semi-opaque, or opaque. In a related embodiment, the first layer is transparent and colorless and the second layer is translucent, semi-opaque, or opaque, and includes one or more pigments, pearls, lakes, or dyes. It has been surprisingly found that color cosmetics according to the invention provide a more natural appearance when a transparent layer is include over or under the pigmented layer,

The first layer may comprises a first optical component and the second layer may comprise a second optical component different from the first optical component. In one implementation, the personal care product may be a product for concealing dark circles under the eyes, wherein the first layer includes an optical blurring agent, and the second layer includes a reflectance pearl. The solid film may be configured to mach the shape of the area under human eyes associated with dark circles, so that when the film is wetted and adhered to the area under the eyes, the appearance of dark circles is reduced.

In another variant, the first layer includes a first cosmetic or therapeutic component and the second layer includes a second cosmetic or therapeutic component that is incompatible with the first cosmetic or therapeutic component. In this manner, combinations of ingredients can be delivered to a human integument that were previously difficult or impossible to deliver together.

In yet another variant, the first and second layers have different dissolution rates in water. For example, the first layer and second layer may constitute opposite faces of the film, and the first layer may have a significantly greater solubility in water than the second layer, such that when the second layer is wetted, the film is capable of adhering to the skin without significantly dissolving said second layer so that it remains in contact with the skin for a prolonged period of time, e.g., one minute or more, more typically, five minutes or more, and preferably, 30 minutes or more. The first layer may include a reconstitutable pigmented cosmetic formulation, such as a foundation, and said second layer may comprise a therapeutic active agent for treating the skin, such as an anti-acne agent or an anti-aging active. After the second layer has been adhered to the skin, the first layer may be rapidly dissolved in water to reconstitute the pigmented cosmetic formulation, which can then be spread over the second layer and the surrounding skin to conceal said second layer while it slowly delivers the thereapeutic agent to the skin.

The advantages of the invention will be most fully realized where the intended cosmetic is one that is traditionally in liquid form, as is the case with many foundations, concealers, lip glosses, and nail polishes. Such liquid cosmetics are difficult to distribute to consumers as samples in magazines, catalogues, and mailers, and no satisfactory solution exists for providing samples of these liquid cosmetics at retailers. However, the invention is not limited to samplers and includes, in its broadest aspect, novel cosmetic forms that will improve the convenience and portability of cosmetics.

In one aspect of the invention, a cosmetic product, including but not limited to a cosmetic sampler, is provided. The cosmetic product includes a sheet-like substrate formed of plastic or paper having a cosmetic formulation deposited on at least a portion of the substrate. Typically, the cosmetic formulation is dry or semisolid because any volatiles have been removed after it is deposited onto the substrate. A layer of a solid water-soluble polymer forms a continuous layer over at least the portion of the substrate that bears the cosmetic formulation to protect the cosmetic formulation from transfer. Alternatively, the cosmetic formulation can be included with the dry water-soluble polymer such that it is homogenously dispersed throughout a matrix formed by the water-soluble polymer. In either case, the layer of dry, water-soluble polymer may be overlaid with a masking layer of tissue or thin plastic, for example, to protect the composition prior to use. In use, the consumer mixes an amount of water to the product sufficient to dissolve the water-soluble polymer and reconstitute the liquid cosmetic.

In another aspect of the invention, a cosmetic sampler sheet is provided. The sampler sheet will have a plurality of sampling areas on a plastic or paper substrate in the form of a sheet and a cosmetic formulation is deposited on each of the sampling areas. In some implementations, the cosmetic formulations will be different at each sample area such that the sample sheet can provide a variety of cosmetics of different shade or other optical attribute. A water-soluble polymeric matrix forms a solid layer over each of the sampling areas and protective mask may be provided to protect the water-soluble polymeric layer. The cosmetic formulations can be reconstituted with a suitable volume of water to produce a liquid cosmetic composition for application to a human integument, such as skin, hair, lashes, eyebrows, and nails. The substrate is typically perforated or scored to permit each sampling area to be readily separated from the others. The sampler sheets can be included in promotional materials, such as magazine or catalogue inserts or as mailers, or may be distributed at retail locations.

Also provided is a method of making a cosmetic sample sheet comprising providing a substrate formed of a sheet of plastic or paper having a plurality of sampling areas defined thereon and depositing cosmetic formulations on each of the sampling areas. Typically, though not necessarily, the cosmetic formulations will be different at each sampling area and will be applied simultaneously to the sheet with multiple filling heads. The cosmetic formulations will typically comprise volatile constituents, such as water, and thus the substrate will be heated to remove the volatile components. After the volatiles are removed from the cosmetic composition to leave a residue thereof on the substrate, a solution or dispersion of water-soluble polymer is applied, typically by spraying, over said sampling areas to cover at least the residue of the cosmetic composition. The sample sheet is then heated to dry and solidify the water-soluble polymer into a coating layer covering the cosmetic formulations. The substrate is typically perforated or scored to permit each sampling area to be readily separated from the others.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### DETAILED DESCRIPTION

The invention concerns personal care products provided in novel forms. As used herein, the term “personal care product” refers to compositions that are intended to be topically applied to a person. Examples of personal care compositions include skin care products, cosmetics, sunscreens, hair care products and the like. As used herein, the term “consisting essentially of” is intended to limit the invention to the specified materials or steps and those that do not materially affect the basic and novel characteristics of the claimed invention, as understood from a reading of this specification.

The term “water-soluble polymer” includes, but is not limited to, polymers with a solubility in water, measured at 25° C., of at least 0.1 gram/liter (g/L). The dissolution rate may be measured by the protocol provided in U.S. Patent Pub. No. 2009/0204341 to Yu et al., the disclosure of which is hereby incorporated by reference. Briefly, a 2-3 mil, 2 cm×1.5 cm, film is weighed. The weight should be about 0.0175 g to about 0.0225 g depending on thickness. The dissolution rate will be affected by thickness, all other factors being equal. The film is placed in the palm of a tester's hand and about 2.5 mL of water are added. With a finger, the wetted film is rubbed in a circular motion, two circles representing a second, until the film is dissolved. The dissolution rate is expressed as the time required to fully dissolve the film.

The water-soluble polymers may be synthetic, natural, or modified polymers derived from natural polymers. Representative water-soluble polymers include wheat or soybean proteins; keratin hydrosylates and sulfonic keratins; casein; albumin; agarose; collagen; glutelin; glucagon; gluten; zein; gelatins and derivatives thereof; polymers derived from chitin or from chitosan, such as anionic, cationic, amphoteric or nonionic chitin or chitosan polymers; polysaccharide polymers such as cellulose-based polymers, for instance cellulose acetate, cellulose acetate phthalate, hydroxyethylcellulose, hydroxypropylcellulose, hydroxymethylcellulose, methylcellulose, ethylhydroxyethylcellulose, hydroxypropylmethylcellulose, hydroxylpropylmethylcellulose phthalate, carboxymethylcellulose and quaternized cellulose derivatives, starches and derivatives thereof, amylose, high amylose starch, hydroxypropylated high amylose starch, and dextrans; acrylic polymers or copolymers such as polyacrylates, polymethacrylates and copolymers thereof; acrylamides such as poly(2-acrylamido-2-methyl-1-propanesulfonic acid); vinyl polymers such as polyvinylpyrrolidones and cross-linked povidone, copolymers of methyl vinyl ether and of maleic anhydride, the copolymer of vinyl acetate and crotonic acid, copolymers of vinylpyrrolidone and of vinyl acetate, copolymers of vinylpyrrolidone and caprolactam, and polyvinyl alcohols; vegatable gums and other natural hydrocolloids such as gum arabic, guar gum, tragacanth gum, xanthan gum and derivatives or karaya gum, alginates, such as sodium alginate, carrageenans, pectin, ulvanes and other algal pullulan, pectin, mannan and galactomannans, glucomannans; mucopolysaccharides including glycoaminoglycans, hyaluronic acid and its derivatives, and chondroitin sulphate; shellac; sandarac gum; dammar resins; elemi gums; copal resins; water soluble waxes; caprolactams; poloxamer; and mixtures and/or derivatives thereof.

Particular mention may be made of modified celluloses, polyacrylic acid and copolymers, polyacrylamides, polyvinyl alcohol and copolymers, vinyl alcohol/vinyl acetate copolymers, polyvinyl pyrrolidone, gelatin, pullan, gaur gum, xanthan gum, gum acacia, polyethylene oxide, natural or synthetic hydrogels, and combinations thereof. Preferably, the water-soluble polymer comprises a cellulosic, such as cellulose esters, alkyl ethers of cellulose, hydroxyalkyl ethers of cellulose, and carboxyalkyl ethers of cellulose. Specific mention may be made of cellulose acetate, cellulose triacetate, cellulose acetate phthalate, hydroxymethylcellulose, hydroxyethylcellulose, hydroxypropylcellulose, methylcellulose, ethylhydroxy ethyl cellulose, hydroxyethyl methylcellulose, hydroxypropylmethylcellulose, hydroxylpropylmethylcellulose phthalate, carboxymethylcellulose, quaternized cellulose derivatives, and combinations thereof. In one embodiment, the water-soluble polymer is a cellulose ether based polymer, polyethylene oxide, or mixtures thereof. In one embodiment, the water-soluble polymer is at least one of methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose.

To make the films, the water-soluble polymer is dissolved or dispersed in a suitable volatile solvent. The volatile solvent may be any solvent that is capable of dissolving or dispersing the water-soluble polymer, but should be one that is topically acceptable in the event that residual amounts remain after drying. The solvent for the water-soluble polymer may be non-polar, polar aprotic, or polar protic, and may be water-miscible or water-immiscible, and includes without limitation, water; acetic acid; ethers such as dimethyl ether, diethyl ether; straight or branched chain alcohols, including methanol, ethanol, propanol, isopropanol and butanol; ketones such as acetone or methyl ethyl ketone; esters such as ethyl acetate; siloxanes; hydrocarbons; toluene, benzene, acetonitrile, dimethylformamide; dimethylsulfoxide; tetrahydrofuran; dichloromethane or chloroform; and combinations thereof. Preferably, the solvent is aqueous and may comprise water or consist essentially of water, by which is meant that other solvents are not intentionally added at level that would measurably affect the vapor pressure or flash point of the solvent system.

Once dissolved or dispersed in the volatile solvent, the water-soluble polymer will comprise from about 0.1% to about 95% of the total weight of the combination, more typically from about 4% to about 70% by weight, and preferably from about 10% to about 50%.

To the dispersion or solution of water-soluble polymer may also be added other agents to modify a characteristic of the resulting matrix, such as its hardness or dissolution time. Suitable thickeners include those described in U.S. Patent Pub. 2010/0204341, to Yu et al., the disclosure of which is hereby incorporated by reference. These include, without limitation, carboxyvinyl polymers, such as the products sold under the names CARBOPOL and PEMULEN (INCI name: Acrylates/C₁₀₋₃₀ alkyl acrylate crosspolymer; available from Noveon), polyacrylates & polymethacrylates, such as the products sold under the names LUBRAJEL and NORGEL (from Guardian) or HISPAGEL (from Hispano Chimica), polyacrylamides, and sodium polyacrylate/dimethicone/cyclopentasiloxane/tri-deceth-6/PEG-PPG-18/18 dimethicone, polyacrylamides, for example, polyacrylamide/C13-C14 isoparaffin/laureth-72-acrylamido-2-methylpropanesulfonic acid polymers and copolymers, poly(2-acrylamido-2-methylpropanesulfonic acid) sold by Clariant (INCI name: ammonium polyacryldimethyltauramide), emulsified crosslinked anionic copolymers of acrylamide and AMPS, such as those sold under the name SEPIGEL 305 (INCI name: Polyacrylamide/C13-14 Isoparaffin/Laureth-7; from Seppic) and under the name SIMULGEL 600 (NCI name: Acrylamide/Sodium acryloyldimethyltaurate copolymer/Isohexadecane/Polysorbate 80; from Seppic), polysaccharide biopolymers, for instance xanthan gum, guar gum, carob gum, acacia gum, scleroglucans, chitin and chitosan derivatives, carrageenans, gellans, alginates, starches, associative polymers, associative polyurethanes, copolymers comprising at least two hydrocarbon-based lipophilic chains comprising from 6 to 30 carbon atoms, separated with a hydrophilic sequence, such as the polyurethanes sold under the names SERAD FX1010, SERAD FX1100 and SERAD FX1035 (from Huls America), RHEOLATE 255, RHEOLATE 278 and RHEOLATE 244 (INCI name: Polyether-urea-polyurethane; from Rheox), DW 1206F, DW 1206J, DW 1206B, DW 1206G, and ACRYSOL RM 2020 (from Rohm & Haas), water-soluble vinyl polymer, and celluloses such as microcrystalline cellulose, carboxymethylcellulose, hydroxymethylcellulose and hydroxypropylcellulose. Thickeners, if present, will typically comprise from about 0.01% to about 50% by weight based on the weight of the water-soluble film-forming polymer.

Plasticizers may also be added, including, without limitation trialkyl citrates, polyols, such as glycerin, propylene glycol, sorbitol, dipropylene glycol, butylenen glycol, pentylene glycol, caprylyl glycol, polyethylene glycols, such as PEG 400, mono-and di-saccharides, and polyetheyleneoxide, to name a few. Propylene glycol is a preferred plasticizer. The plasticizer, if present, will typically comprise from about 0.01% to about 50% by weight based on the weight of the water-soluble film-forming polymer.

The films of the invention may be prepared according to known methods. By way of illustration, an homogenous dispersion of water-soluble polymer in water may be formed which includes the water-soluble polymer at about 10% to about 50% by weight of the dispersion. Other components, including, without limitation, plasticizers, thickeners, emollients, humectants, emulsifiers, colorants, and active agents may be added in any order and homogenously dispersed. The solution or suspension may be cast, painted, sprayed, extruded, enrobed or otherwise deposited onto a surface, to form a film or desired thickness between about 0.1 mils to about 20 mils in thickness (the term “mil” refers to 1/1000 of an inch). After the suspension has been cast, the volatiles, including water, are removed by evaporation or by heating to solidify the water-soluble polymer into a solid film. Multilayered films may be made by allowing the first layer to solidify before casting a second layer thereon. The second layer may be cast or otherwise applied directly on the first. The dried films may then be cut into any desired shape.

The formulations may include essentially any topically acceptable ingredient found in personal care products provided that they do not adversely affect the ability of the film to harden. “Topically acceptable” refers to ingredients that are generally regarded as safe for application to a human integument, including hair of the scalp, eyelashes, eyebrows, skin of the face, lips, nails, etc. The topically acceptable ingredients will include, without limitation, colorants (e.g., pigments, pearls, lakes, and dyes), fillers, film formers (water-resistant and/or oil resistant), emollients, humectants, emulsifiers, thickeners, rheology modifiers, chelating agents, pH adjusters, skin conditioning agents, active (therapeutic) agents, sunscreens, sunblocks, fragrances, preservatives, and the like. Any ingredient that is customarily found in the particular topical formulation is contemplated to be suitable. Jojoba hydrate, which is a mixture of jojoba alcohol, potassium jojobate, and butylene glycol from Desert Whale, provides good aesthetic properties to the film.

The personal care products may be cosmetic compositions, and in particular color cosmetics which, by definition, comprise colorants. The colorants may comprise from about 0.1% to about 75% by weight of the dry film, more typically, from about 1% to about 50% by weight of the film. The colorants may be, without limitation, pigments, lakes, or dyes. There is essentially no limitation on the nature of the pigments, lakes, or dyes and include those listed in the International Cosmetic Ingredient Dictionary and Handbook, 12^{th} Ed. (2008), incorporated by reference herein.

The term “pigment” refers to an inorganic or organic particle that is insoluble in the aqueous base of the cosmetic and is intended to impart color or hiding power. Preferred pigments are inorganic pigments, such as metal oxides and hydroxides, particular mention being made of magnesium oxide, magnesium hydroxide, calcium oxide, calcium hydroxides, aluminum oxide, aluminum hydroxide, chromium oxide, chromium hydroxide, cobalt oxide, iron oxides, iron hydroxides manganese oxides, nickel oxide, tin oxide, titanium dioxide, zirconium oxide, and zinc oxide to name but a few. Other suitable pigments include ultramarine blue (i.e., sodium aluminum silicate containing sulfur), Prussian blue, manganese violet, sericite, potassium ferricyanide, potassium ferrocyanide, potassium ferrocyanide trihydrate, magnesium carbonate, calcium carbonate, silica, talc, mica, magnesium silicate, aluminum magnesium silicate, carbon black, and composite oxides and composite hydroxides such as iron titanate, cobalt titanate and cobalt aluminate to name a few. For the purposes of this invention, fillers which impart hiding power, including talc, calcium carbonate, and silica, may also be considered pigments.

Organic pigments can include, but are not limited to, carmine, phthalocyanine blue and green pigment, diarylide yellow and orange pigments, and azo-type red and yellow pigments such as toluidine red, litho red, naphthol red and brown pigments, and combinations thereof.

Lakes generally refer to a colorant prepared from a water-soluble organic dye (e.g., D&C or FD&C) which has been precipitated onto an insoluble reactive or adsorptive substratum or diluent. The term “D&C” means drug and cosmetic colorants that are approved for use in drugs and cosmetics by the FDA. The term “FD&C” means food, drug, and cosmetic colorants which are approved for use in foods, drugs, and cosmetics by the FDA. Certified D&C and FD&C colorants are listed in 21 C.F.R. §74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Substrates suitable for forming lakes include, without limitation, mica, bismuth oxychloride, sericite, alumina, aluminum, copper, bronze, silver, calcium, zirconium, barium, and strontium, titanated mica, fumed silica, spherical silica, polymethylmethacrylate (PMMA), micronized Teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, nylon, silica silylate, silk powder, sericite, soy flour, tin oxide, titanium hydroxide, trimagnesium phosphate, walnut shell powder, and mixtures thereof. Suitable lakes include, without limitation, those of red dyes from the monoazo, disazo, fluoran, xanthene, or indigoid families, such as Red 4, 6, 7, 17, 21, 22, 27, 28, 30, 31, 33, 34, 36, and Red 40; lakes of yellow pyrazole, monoazo, fluoran, xanthene, quinoline, dyes or salt thereof, such as Yellow 5, 6, 7, 8, 10, and 11; lakes of violet dyes including those from the anthroquinone family, such as Violet 2, as well as lakes of orange dyes, including Orange 4, 5, 10, 11, and the like. Suitable Lakes of D&C and FD&C dyes are defined in 21 C.F.R. §82.51.

The pigments may be surface-modified with hydrophobic or hydrophilic coatings to enhance or alter one or more properties such as their optical properties, dispersibility, or substantivity with the integument, examples including the AEROSIL™ line of hydrophobically modified fumed silica particles from Degussa Corporation of Parsippany, N.J., and the AEROXIDE™ ALU C805 hydrophobically modified alumina from Evonik Industries, as well as the COVALUMINE™ line of pigments marketed by SENSIENT™ Cosmetic Technologies LCW which comprise pigments bonded to a platelet-shaped alumina substrate and a triethoxycaprylylsilane surface treatment covering the particle at about 1% by weight. The COVALUMINE™ range includes, but are not limited to, Alumina/Titanium Dioxide/Triethoxycaprylylsilane 1% (COVALUMINE™ Atlas White AS), Alumina/D&C Red Aluminum Lake CTD/Triethoxycaprylylsilane 1% (COVALUMINE™ Red Rose AS), Alumina/D&C Red Aluminum Lake CTD/Triethoxycaprylylsilane 1% (COVALUMINE™ Sonoma Red AS), Alumina/Black Iron Oxide CTD/Triethoxycaprylylsilane 1% (COVALUMINE™ Sonoma Black AS), Alumina/D&C Red #6 Aluminum Lake CTD/Triethoxycaprylylsilane 1% (COVALUMINE™ Fire Red AS), Alumina/Yellow Iron Oxide CTD/Triethoxycaprylylsilane 1% (COVALUMINE™ Sonoma Yellow AS), Alumina/D&C Blue #1 Aluminum Lake CTD/Triethoxycaprylylsilane 1% (COVALUMINE™ Astral Blue AS), Alumina/Carmine CTD/Triethoxycaprylylsilane 1% (COVALUMINE™ Campari AS), Alumina/Yellow #5 CTD/Triethoxycaprylylsilane 1% (COVALUMINE™ Sunburst AS), Alumina/Triethoxycaprylylsilane 1%, and combinations thereof, each of which is available from SENSIENT™ Cosmetic Technologies LCW.

The formulations may also comprise an interference or pearling agent, alone or in combination with pigment and other colorants. Interference or pearl pigments are typically comprised of micas layered with about 50 to 300 nm films of TiO₂, Fe₂O₃, or Cr₂O₃ or the like. These include white nacreous materials, such as mica covered with titanium oxide or covered with bismuth oxychloride; and colored nacreous materials, such as titanium mica with iron oxides, ferric blue, chromium oxide, or organic pigment. The pearling agents will typically be titanated micas or bismuth oxychloride. Bismuth oxychloride pearls may also be bonded to colored pigments to vary the hue, as in the CHROMA-LITE™ line from Englehard Corporation (Iselin, N.J.), which is a combination of a colored pigment bonded to bismuth oxychloride and mica using calcium stearate. The pearl can also include a natural nacre. Also suitable are terephthalates used as glitter agents, such as polyethylene terephthalate.

The composition can include fibrous pigments. In certain embodiments, the fibrous pigments have an average diameter between about 0.05 and 20 microns and an average aspect ratio of 5 or greater. Suitable fibrous pigments may be comprised of organic substances, inorganic substances of mixtures thereof. Organic fibers include, but are not limited to, Nylon, Polyacrylic acid (PAA), Cross-linked polyethylene (PEX or XLPE), Polyethylene (PE), Polyethylene terephthalate (PET or PETE), Polyphenyl ether (PPE), Polyvinyl chloride (PVC), Polyvinylidene chloride (PVDC), Polylactic acid (PLA), Polypropylene (PP), Polybutylene (PB), Polybutylene terephthalate (PBT), Polyamide (PA), Polyimide (PI), Polycarbonate (PC), Polytetrafluoroethylene (PTFE), Polystyrene (PS), Polyurethane (PU), Polyester (PEs), Acrylonitrile butadiene styrene (ABS), Polymethyl methacrylate (PMMA), Polyoxymethylene (POM), Polysulfone (PES), Styrene-acrylonitrile (SAN), Ethylene vinyl acetate (EVA), Styrene maleic anhydride (SMA) and polyacrylonitrile; or polyvinyl alcohol (PVA). The fibrous pigments also may take the form of a polymeric fiber coated with a metal oxide, such as TiO₂, iron oxides, or the like as described, for example, in Drew et al. “Metal oxide coated polymer nanofibers,” Nano Letters 2003, Vol. 3, No. 2, pp, 143-147). The fibrous pigments may be composed of inorganic oxides, including but not limited to titania, for example titanium dioxide (TiO₂); iron oxides, for example FeO and Fe₂O₃; alumina or aluminum oxides, for example Al₂O₃; zinc oxide (ZnO), silica (SiO₂), and combinations thereof. In a particular embodiment, the fibrous pigment is solid TiO₂, for example solid TiO₂ nanofiber prepared by electrospinning methods described, for example, in J. Y. Park and S. S. Kim, Metals and Materials International, Vol 15(1), pp. 95-99 (2009). The fibrous pigment may comprise a clay mineral compound or aluminasilicate including but not limited to Halloysite. In other embodiments, the fibrous pigment is hollow or in the shape of a tube. In a preferred embodiment the fibrous pigment is Halloysite, which is hollow or tube-shaped, optionally filled with one or more substances which may provide additional benefits to the skin, for example moisturizing compounds such as glycerin.

The formulations can also comprise other particulates including, without limitation, kaolin, Nylon powders such as Orgasol™, polyethylene powder, Teflon™ powder, starch, boron nitride, cross-linked polymethacrylate copolymers powders such as Polytrap™ (Dow Corning), polymeric microspheres such as Expancel™ (Nobel industries), silicone resins microbeads such as Tospearl™ from Toshiba), and combinations thereof. Other suitable particulates include the particulate silicon wax sold under the trade name Tegotop™ 105 (Degussa/Goldschmidt Chemical Corporation) and the particulate vinyl polymer sold under the name Mincor™ 300 (BASF).

The formulation may also include a goniochromatic coloring agent, by which is meant a coloring agent whose color changes with the angle of observation. The goniochromatic coloring agent may be, for example, multilayer interference structures and liquid-crystal coloring agents. Suitable goniochromatic coloring agent are disclosed in L'Oreal's U.S. Pat. No. 7,767,214, the disclosure of which is hereby incorporated by reference, as briefly described below.

Goniochromatic multilayer interference structures may comprise two or more layers, which may be the same or different, composed of various metal and oxides. The multilayer structure may or may not be symmetrical in the composition of the stacked layers. Multilayer interference structures include, without limitation, Al/SiO₂/Al/SiO₂/Al; Cr/MgF₂/Al/MgF₂/Cr; MoS₂/SiO₂/Al/SiO₂/MoS₂; Fe₂O₃/SiO₂/Al/SiO₂/FeO₃; Fe₂O₃/SiO₂/Fe₂O₃/SiO₂/Fe₂O₃; MoS₂/SiO₂/mica-oxide/SiO₂/MoS₂; Fe₂O₃/SiO₂/mica-oxide/SiO₂/Fe₂O₃; TiO₂/SiO₂/TiO₂; TiO₂/Al₂O₃/TiO₂; SnO/TiO₂/SiO₂/TiO₂/SnO; Fe₂O₃/SiO₂/Fe₂O₃; SnO/Mica/TiO₂/SiO₂/TiO₂/Mica/SnO; silica/titanium oxide/tin oxide; silica/brown iron oxide; and silica/titanium oxide/mica/tin oxide. Suitable multilayered interference pigments are sold under the name Chromaflair™ by Flex; Sicopearl™ by BASF; Xirona™ by Merck (Darmstadt); and Infinite Colors™ from Shiseido.

Goniochromatic coloring agents with a multilayer structure comprising an alternation of polymer layers, for example of the type such as polyethylene naphthalate and polyethylene terephthalate, may also be used, including those described in WO-A-96/19347 and WO-A-99/36478, the disclosure of which are hereby incorporated by reference. Goniochromatic coloring agents with a polymeric multilayer structure are available from 3M under the name Color Glitter™.

The composition may also comprise goniochromatic interference fibers having a multilayer structure. Fibers with a multilayer structure of polymers are described especially in EP-A-921,217, EP-A-686,858 and U.S. Pat. No. 5,472,798, the disclosures of which are hereby incorporated by reference, The multilayer structure may comprise at least two layers made of at least one synthetic polymer having a refractive index ranging from 1.30 to 1.82. The polymers that are preferred for making the fibers are polyesters such as polyethylene terephthalate, polyethylene naphthalate, polycarbonate; acrylic polymers such as polymethyl methacrylate; polyamides. Goniochromatic fibers with a polyethylene terephthalate/nylon-6 two-layer structure are sold by Teijin under the name Morphotex.

The goniochromatic coloring agent also may comprise liquid-crystal coloring agents. The liquid-crystal coloring agents comprise, for example, silicones or cellulose ethers onto which are grafted mesomorphic groups. Examples of liquid-crystal goniochromatic particles that may be used include, for example, those sold by Chenix and also the products sold under the name Helicone® HC by Wacker.

Reflective pigments may also be included. Reflective pigments provide a high specular reflection of visible light. Reflective pigments include those wherein a substrate having a particle size from about 10 μm to about 250 μm, in platelet, globular, or spherical form, is partially or completely coated with a non-matt and non-scattering surface layer of a metal or metal oxide. The substrate may be chosen from glasses, ceramics, graphite, metal oxides, aluminas, silicas, silicates, especially aluminosilicates and borosilicates and synthetic mica (e.g., fluorophlogopite), to name a few. The metal or metal oxide may be, without limitation, titanium oxides, iron oxides, tin oxide, chromium oxide, barium sulfate, MgF₂, CeF₃, ZnS, ZnSe, SiO₂, Al₂O₃, MgO, Y₂O₃, SeO₃, SiO, HfO₂, ZrO₂, CeO₂, Nb₂O₅, Ta₂O₅ and MoS₂, and mixtures thereof. Reflective pigments preferably have a spectral reflectance in the visible spectrum of at least 70%.

Other reflective pigments include those having non-goniochromatic layered structure of two or more polymeric and/or metallic layers of different refractive indices. For example, reflective particles comprising layers of 2,6-polyethylene naphthalate (PEN) and of polymethyl methacrylate are sold by 3M under the name Mirror Glitter™. Other effect pigments are available under the trade name Metasomes Standard/Glitter in various colours (yellow, red, green, blue) from Flora Tech.

Optical brighteners may also be included. Suitable optical brighteners are disclosed in U.S. Pat. No. 6,313,181, the disclosure of which is hereby incorporated by reference. These include derivatives of stilbene and 4,4′-diaminostilbene, e.g., bistriazinyl derivatives; derivatives of benzene and biphenyl, e.g., styryl derivatives; pyrazolines, bis(benzoxazol-2-yl) derivatives, coumarins, carbostyrils, naphthalimides, s-triazines, pyridotriazoles, and the like. The optical brighteners or fluorescent brighteners may also be an inorganic fluorescent glass, such as are described in U.S. Pat. Nos. 5,635,109, and 5,755,998, the contents of which are incorporated herein by reference. Preferably the optical brightener shows a green or blue fluorescence.

The compositions can also include a spherical scattering component comprising spherical powders that achieve a soft focus look such as calcium aluminum borosilicate, PMMA, polyethylene, polystyrene, methyl methacrylate crosspolymer, nylon-12, ethylene/acrylic acid copolymer, boron nitride, Teflon, silica, or the like.

The compositions may include non-volatile solvents and oils including without limitation, fatty alcohols, fatty acid esters, lanolin, hydrocarbon oils, silicone oils, and mixtures thereof.

The compositions may also include film forming agents selected from the group consisting of waxes, water-soluble polymeric film formers, and water-insoluble polymeric film formers. “Film forming agents” are well known and comprise a group of chemicals that leave a pliable, cohesive, and continuous covering over an integument when applied on its surface. Polymeric film forming agents include, without limitation, cellulosics, polyolefins, polyvinyls, polyacrylates, polyurethanes, silicones, silicone acrylates, polyamides, polyesters, fluoropolymers, polyethers, polyacetates, polycarbonates, polyimides, epoxys, formaldehyde resins, and homopolymers and copolymers of any of the foregoing. In other embodiments, the topical compositions will be free of wax and/or free of water-insoluble polymeric film formers.

The compositions may comprise one or more volatile solvents to facilitate their application to the substrate through fill nozzles, droppers, or other expedient for meting the formulation onto an area of the substrate. The volatile solvents may be, without limitation, water, C₃-C₆ ketones (e.g., acetone), C₃-C₆ esters (e.g., ethyl acetate), C₂-C₆ alcohols (e.g., ethanol), silicones (e.g., trimethicone, cyclomethicone pentamer, etc.), hydrocarbons (e.g., isododecane), or combinations thereof. Because the volatiles are removed after the topical formulation is applied to the substrate, their identity is not critical. The topical formulation may be applied to a specific area of the substrate, or may cover the major portion of a side of the substrate, or may cover an entire side of the substrate.

A layer of a water-soluble polymer is then deposited over at least a portion of the substrate, including the portion on which the residue of the topical formulation is deposited. The term “water-soluble polymer” includes polymers with a solubility, measured at 25° C., of at least 0.1 gram/liter (g/L), but preferably at least 1.0 g/L. The water-soluble polymers should be topically acceptable, as they will ultimately be dissolved and included in the final formulation that is applied to a human integument.

Any other components typically associated with the intended cosmetic can also be added to the water-soluble polymeric solution or suspension, provided that these additional components do not adversely affect the solid film formed thereby. These include, without limitation, any of the detackifiers, conditioners, and emollients listed in U.S. Patent Pub. 2010/0204341, to Yu et al., the disclosure of which is hereby incorporated by reference; as well as colorants, preservatives, pH adjustors, reducing agents, fragrances, foaming agents, tanning agents, depilatory agents, flavors, astringents, antiseptics, deodorants, antiperspirants, insect repellants, sunscreens, sun blocks, bleaches and lighteners, anti-dandruff agents, adhesives, polishes, strengtheners, fillers, barrier materials, and biocides.

In one embodiment of the invention, the compositions may include additional skin actives, including but not limited to, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, and advanced glycation end-product (AGE) inhibitors.

The composition may comprise additional active ingredients having anti-aging benefits, as it is contemplated that synergistic improvements may be obtained with such combinations. Exemplary anti-aging components include, without limitation, botanicals (e.g., Butea Frondosa extract); phytol; thiodipropionic acid (TDPA) and esters thereof; retinoids (e.g., 9-cis retinoic acid, 13-cis retinoic acid, all-trans retinoic acid and derivatives thereof, phytanic acid, retinol (Vitamin A) and esters thereof, such as retinol palmitate, retinol acetate and retinol propionate, and salts thereof and others); hydroxy acids (including alpha-hydroxyacids and beta-hydroxyacids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof, etc.), to name a few. Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof.

Suitable organic sunscreens include, without limitation, benzophenone-3 (oxybenzone), dioxybenzone, menthyl anthranilate, para aminobenzoic acid (PABA), octyl methoxycinnamate (octinoxate), octocrylene, drometrizole trisiloxane, octyl salicylate (octisalate), homomenthyl salicylate (homosalate), octyl dimethyl PABA, butylmethoxy dibenzoylmethane (avobenzone), 4-methyl benzilidene camphor, octyl triazone, ethyl PABA, hydroxy methylphenyl benzotriazole, methylene bisbenzotriazoyl-tetramethylbutylphenol, bis-ethythexyloxyphenol methoxyphenol triazine, and mixtures thereof.

Suitable inorganic sunscreens include titanium dioxide and zinc oxide. In one embodiment, the film will include zinc oxide, preferably and optically transparent dispersion of zinc oxide particles. In one embodiment, the dispersion of zinc oxide particles has a median particle diameter (D₅₀) between about 1 micron (μm) and about 5 μm and a D₉₀ of less than about 10 μm, on a volume basis. In another embodiment, the dispersion of zinc oxide particles has a D₅₀ between about 1.5 μm and about 3 μm; a D₉₀ of less than about 6.5 μm; and a D₁₀ greater than 0.1 μm, on a volume basis. A suitable transparent dispersion of zinc oxide particles is available from Dow as Zinclear™ IM. The zinc oxide particles may comprise from about 1% to about 30% of the weight of the film.

The compositions of the invention may optionally comprise other active and inactive ingredients typically associated with cosmetic and personal care products, including, but not limited to, excipients, fillers, emulsifying agents, antioxidants, surfactants, film formers, chelating agents, gelling agents, thickeners, emollients, humectants, moisturizers, vitamins, sodium ascorbyl/cholesteryl phosphate, minerals, botanicals, viscosity and/or rheology modifiers, sunscreens, keratolytics, depigmenting agents, retinoids, hormonal compounds, alpha-hydroxy acids, trioxaundecanedioic acid, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, staining agents, depigmenting agents, hypopigmenting agents, preservatives, stabilizers, pharmaceutical agents, photostabilizing agents, and mixtures thereof.

A personal care product of the invention will have the form of a solid film. The film may comprise a singular layer or may comprise plural laminated layers, by which is meant two or more layers facially joined, preferably in superposition. The first layer will include a first solid water-soluble polymeric matrix, and the second layer will include a second solid water-soluble polymeric matrix. The polymeric matrix material may be different in each layer, or may be different in two or more layers, or may be the same in each layer. In one embodiment, the dissolution rate will be different for at least two of the layers. Each layer may have a different optical property. For example, each layer may have a different refractive index, a different light transmittance, or different hue or optical effect. One layer may be transparent and another layer may be translucent, semi-opaque, or opaque. One layer may be transparent and another layer may be pigmented, for example, with a pigment such as iron oxides and/or titanium dioxide. Any of the layers may comprise a skin benefit agent, such as a therapeutic skin active.

In one embodiment, the personal care product may be a product for concealing dark circles under the eyes, wherein the first layer includes an optical blurring agent, and the second layer includes a reflectance pearl. The solid film may be configured to match the shape of the area under human eyes associated with dark circles, so that when the film is wetted and adhered to the area under the eyes, the appearance of dark circles is reduced.

In another embodiment, the first layer includes a first cosmetic or therapeutic component and the second layer includes a second cosmetic or therapeutic component that is incompatible with the first cosmetic or therapeutic component. In this manner, combinations of ingredients can be delivered to a human integument that were previously difficult or impossible to deliver together. For example, the first and second layers may each have different pH's which allow them to stabilize or contain two compounds that are not stable at the same pH. The first and second layers may also comprise components that chemically react when brought into contact with one another.

In another embodiment, the first and second layers have different dissolution rates in water and may constitute opposite faces of the film. In one embodiment, the first layer may have a significantly greater solubility in water than the second layer, such that when the second layer is wetted, the film is capable of adhering to the skin without significantly dissolving said second layer. Preferably, the second layer will adhere to the skin so and remains in contact with the skin for a prolonged period of time, e.g., one minute or more, more typically, five minutes or more, and preferably, 30 minutes or more. The first layer may include a reconstitutable pigmented cosmetic formulation, such as a foundation, and the second layer may comprise a therapeutic active agent for treating the skin, such as an anti-acne agent or an anti-aging active. After the second layer has been adhered to the skin, the first layer may be rapidly dissolved in water to reconstitute the pigmented cosmetic formulation, which can then be spread over the second layer and the surrounding skin to conceal said second layer while it slowly delivers the thereapeutic agent to the skin. The product may be applied to a blemish or comedome and the second layer will adhere to the skin over the blemish or comedome and slowly deliver an active agent to treat the skin, while the first layer is readily dissolvable and reconstituted to a concealor that covers the area.

In use, the consumer will add water to the water-soluble film, for example by applying water drop-wise onto the surface, to wet the film. The wetted film will then adhere to a human integument, including the hair of the scalp, eye brows, eye lashes, skin of the face or body, lips, nails, etc.

The personal care products of the invention may optionally include a substrate composed of plastic or paper (cellulosics) to support and protect the films. The substrate takes the form of a sheet by which is mean that it is elongated in two dimensions but is essentially flat, having a thickness between about 0.1 and about 20 mils. The sheet will ideally be flexible and sufficiently resistant to water and other volatile solvents, such as alcohols, lower hydrocarbons, and silicone fluids, such that it is not deformed or damaged when a liquid dispersion is applied to its surface.

A masking layer of tissue or plastic may be place over top of the water-soluble polymeric film to protect the film until the product is ready for use, although its use is strictly optional. The masking layer, if used, should be readily pealable from the water-soluble polymer layer and will ideally be transparent so that the the cosmetics can be seen through the mask.

All percentages are by weight, based on the total weight of the composition, unless otherwise indicated.

All references including patent applications and publications cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A personal care product, having the form of a solid film comprising a water-soluble polymeric matrix having an optical component homogenously dispersed therein, said optical component being selected from the group consisting of liquid crystals, glitters, optical brighteners, cosmetic fibers, and combinations thereof, wherein said solid film is capable of being wetted and adhered to a human integument.

2. A personal care product, having the form of a solid film comprising a water-soluble polymeric matrix, wherein said solid film is capable of being wetted and adhered to a human integument; and wherein said film includes a polymeric film former that shrinks on drying such that the film contracts in one or more dimensions when dried on the skin to thereby provide a tightening effect to the skin.

3. A personal care product, having the form of a solid, transparent film comprising a water-soluble polymeric matrix having an optically transparent dispersion of zinc oxide particles homogenously dispersed therein, wherein said solid film is capable of being wetted and adhered to a human integument to leave a transparent film thereon.

4. The personal care product according to claim 3, wherein said dispersion of zinc oxide particles has a median particle diameter (D₅₀) between about 1 micron (μm) and about 5 μm and a D₉₀ of less than about 10 μm, on a volume basis.

5. The personal care product according to claim 3, wherein said dispersion of zinc oxide particles has a D₅₀ between about 1.5 μm and about 3 μm; a D₉₀ of less than about 6.5 μm; and a D₁₀ greater than 0.1 μm, on a volume basis.

6. The personal care product according to claim 3, wherein said zinc oxide particles comprise from about 1% to about 30% of the weight of said film.

7. The personal care product according to any of claims 1-3, wherein said water-soluble polymeric matrix comprises a polymer selected from the group consisting of hydroxyethyl cellulose, hydroxypropyl cellulose, hydroxypropylmethyl cellulose, carboxymethyl cellulose, sodium carboxymethyl cellulose, methyl cellulose, polyvinyl pyrrolidone, polyvinyl alcohol, sodium alginate, polyethylene glycol, polyethylene oxide, xanthan gum, tragacanth gum, guar gum, acacia gum, gum arabic, pullan, carrageenan, polyacrylic acid, methyl(meth)acrylate copolymers, carboxyvinyl copolymers, and combinations thereof.

8. The personal care product according to claim 7, wherein the water soluble film forming agent is hydroxypropyl methylcellulose.

9. The personal care product according to claim 7, further including a plasticizer in an amount from about 0.01% to about 40.0% of the weight of said film, and a thickener in an amount from about 0.01% to about 10.0% of the weight of said film.

10. The personal care product according to any of claims 1-3, wherein said film is between about 1 and about 5 mils in thickness.

11. A personal care product, having the form of a solid film comprising plural laminated layers, including a first layer of a first solid water-soluble polymeric matrix, and a second layer of a second solid water-soluble polymeric matrix, wherein said solid film is capable of being wetted and adhered to a human integument.

12. The personal care product according to claim 11, wherein said first and second layers have different refractive indices.

13. The personal care product according to claim 12, wherein said first layer is transparent and said second layer is translucent, semi-opaque, or opaque.

14. The personal care product according to claim 13, wherein said first layer is transparent and colorless and said second layer is translucent, semi-opaque, or opaque, and includes one or more pigments, pearls, lakes, or dyes.

15. The personal care product according to claim 11, wherein said first layer comprises a first optical component and said second layer comprises a second optical component different from said first optical agent.

16. The personal care product according to claim 15, wherein said first layer includes an optical blurring agent, and said second layer includes a reflectance pearl, and wherein said solid film is configured to match the shape of the area under human eyes associated with dark circles, wherein the film is capable of being wetted and adhered to the area under the eyes to reduce the appearance of dark circles.

17. The personal care product according to claim 11, wherein said first layer includes a first cosmetic or therapeutic component and said second layer comprises a second cosmetic or therapeutic component that is incompatible with said first cosmetic or therapeutic component.

18. The personal care product according to claim 11, wherein said first and second layers have different dissolution rates in water, wherein said solid film is capable of being wetted and adhered to a human integument.

19. The personal care product according to claim 18, wherein said first layer and second layer constitute opposite faces of said film, and said first layer has a greater solubility in water than said second layer, wherein said first layer includes a reconstitutable pigmented cosmetic formulation and said second layer comprises a therapeutic active agent for treating the skin, such that when the second layer is wetted, the film is capable of adhering to the skin without significantly dissolving said second layer, and said first layer is capable of rapid dissolution in water to reconstitute the pigmented cosmetic formulation, such that the second layer may deliver said therapeutic active agent to the skin over a prolonged duration, while said pigmented cosmetic formulation may be spread over the second layer and skin to conceal said second layer.

20. The personal care product according to claim 19, wherein said first layer has a dissolution rate in water faster than 15 seconds, and said second layer has a dissolution rate in water of slower than one minute.

21. The personal care dissolvable film of claim 11, wherein said first and second water-soluble polymeric matrices comprises a polymer independently selected from the group consisting of methylcellulose, hydroxypropyl methylcellulose, hydroxyethyl cellulose, cationic hydroxyethyl cellulose, hydrophobically modified hydroxyethyl cellulose, or cationic hydrophobically modified hydroxyethyl cellulose.
